(19) United States
(12) Patent Application Publication
(10) Pub. No.: US 2022/0001375 A1
(43) Pub. Date: Jan. 6, 2022

(54) CONTAINER AND KIT FOR PREPARING CELL CULTURE IN HIGH-CLEANLINESS SPACE

(71) Applicant: TERUMO KABUSHIKI KAISHA, Tokyo (JP)

(72) Inventors: Eri NOGUCHI, Kanagawa (JP); Risa YUKI, Kanagawa (JP)

(73) Assignee: TERUMO KABUSHIKI KAISHA, Tokyo (JP)

(21) Appl. No.: 17/478,028

(22) Filed: Sep. 17, 2021

Related U.S. Application Data
(63) Continuation of application No. PCT/JP2020/012938, filed on Mar. 24, 2020.

(30) Foreign Application Priority Data
Mar. 25, 2019 (JP) 2019-057131

(51) Int. Cl. B01L 1/02; B25J 21/02

(52) U.S. Cl. CPC B01L 1/025; B25J 21/02

(57) ABSTRACT

A device enables easy preparation of a cell culture in a clean environment without introducing a large-scale facility for preparing a high-cleanliness space in the preparation of a cell culture. The device includes a sealed pouch-shaped container including: an internal space having a cleanliness level kept within a range from Class 1 to 9 specified in ISO 14644-1 by an air filter; an operation section that enables operation inside the container from outside the container; and a storage section that stores various materials required for preparing a cell culture.

### CROSS-REFERENCES TO RELATED APPLICATIONS

This application is a continuation of International Patent Application No. PCT/JP2020/012938 Filed on Mar. 24, 2021, which claims priority to Japanese Patent Application No. 2019-057131 filed on Mar. 25, 2021, the entire content of both of which is incorporated herein by reference.

### TECHNOLOGICAL FIELD

The present disclosure relates to a sealed pouch-shaped container for preparing a cell culture. The container includes materials used for preparing a cell culture and has a clean interior.

### BACKGROUND DISCUSSION

In recent years, attempts have been made to transplant various cells for the repair of damaged tissues and the like. For example, fetal cardiomyocytes, skeletal myoblasts, mesenchymal stem cells, cardiac stem cells, ES cells, and iPS cells are used for experiments to repair myocardial tissues damaged by ischemic heart disease such as angina pectoris and myocardial infarction (Haraguchi et al., Stem Cells Transl Med. 2012 February; 1(2): 136-41).

Such experiments lead to the development of, for example, sheet-shaped cell cultures obtained by forming cells into a sheet. Applications of such cell cultures have partially entered the stage of clinical application, and cell cultures have already been used in regenerative medicine and the like in medical settings (HeartSheet (registered trademark) package insert (revised in February 2019)).

In the preparation of a cell culture in clinical application, it is necessary to work in a high-cleanliness space such a sterile area (HeartSheet (registered trademark) package insert (revised in February 2019)). For example, Japanese Patent No. 5592080 discloses a cell culture system that reduces the risk of contamination in a sterile area. Japanese Patent Application Publication No. 2010-259378 discloses a cell culture system that reduces the risk of cross-contamination. However, it is necessary to introduce a large-scale facility such as an isolator in both systems. Furthermore, in the system disclosed in Japanese Patent No. 5592080, when a cell culture is introduced into a centrifuge or an incubator, the cell culture remains at risk of passing through a non-sterile area. With regard to the system disclosed in Japanese Patent Application Publication No. 2010-259378, a device such as a centrifuge is installed in a sterile area, and damages are accumulated on the device during sterilization. Japanese Patent Application Publication No. 2001-70402 discloses a system that does not use a large-scale facility. However, the function of this system is to thaw frozen cells but not to culture cells.

### SUMMARY

With the progress of clinical application of cell cultures for transplantation, treatments using cell cultures have been demanded more widely. Such cell cultures for transplantation need to be prepared in hospitals where transplantation surgery is performed. Therefore, in order to use the cell cultures in clinical application, hospitals are required to have a space with a cleanliness level kept high for preparing these cell cultures. The preparation of such a space requires the introduction of large facilities, which requires a heavy capital investment. The introduction of such facilities and the handling of cell cultures for transplantation have been limited to some highly specialized hospitals. In the preparation of a cell culture, objects of the present invention are to minimize a large-scale facility and to enable simple preparation of a cell culture in a clean environment.

The present inventors have found that, in the preparation of a cell culture, instead of a large facility such as an isolator or a safety cabinet which has been used in the past, a sealed pouched container having an internal space with a cleanliness level enhanced by an air filter or the like makes it possible to maintain a high-cleanliness space for sufficiently preparing a cell culture. The present inventors have found that preparing a cell culture inside the container enables the preparation of a cell culture in a clean space without introducing a large-scale facility.

The disclosure here relates to the following techniques.

[1] A container sealed and having a pouch shape, the container including: an air filter configured to maintain a space inside the container to have a cleanliness level within a predetermined range of Class 1 to 9 specified in ISO 14644-1; at least one storage section housed inside the container, being configured to store a material to be used for preparing a cell culture; and an operation section configured to allow operation of the material to be used for preparing a cell culture from outside the container.

[2] The container according to [1], in which the container has a collapsible and expandable structure.

[3] The container according to [1] or [2], in which the container is made of a freezable material.

[4] The container according to any one of [1] to [3], in which the operation section is a glove-shaped structure protruding into the container.

[5] The container according to any one of [1] to [4], in which the storage section includes at least one selected from the group consisting of a cell culture section configured to store a seeded cell and to culture the cell, a liquid storage section configured to store a plurality of liquids, a centrifugation section configured to store a cell suspension to be centrifuged, a cell preservation section configured to store a cell to be cultured, and a waste liquid section configured to store a waste liquid.

[6] The container according to any one of [1] to [5], in which the storage section has a structure separable from the container.

[7] The container according to [5] or [6], in which the cell culture section includes a temperature-responsive cell culture substrate.

[8] A kit for preparing a cell culture, the kit including the container according to any one of [1] to [7].

[9] The kit for preparing a cell culture according to [8], the kit further including a culture vessel having a temperature-responsive cell culture substrate.

[10] The kit for preparing a cell culture according to [8] or [9], the kit further including an instrument used for preparing a cell culture.

[11] According to another aspect, a method for preparing a cell culture inside a container from outside the container comprises: seeding a cell to be cultured in a space inside the container while the container is sealed, with the container being a pouch-shaped container and the space inside the container having a cleanliness level kept within a predetermined range specified in ISO 14644-1 during the seeding of the cell; and culturing the cell in the space in the container.

[12] The method for preparing a cell culture according to [11], the method including a) warming the container having a pouch shape, b) placing the container in a bucket of a centrifuge, or c) setting up the container in an incubator when the container is in a deflated state.

[13] The method for preparing a cell culture according to [11] or [12], the method including allowing an operation section and each storage section to be used when the container having a pouch shape is in an inflated state.

[14] The method according to any one of [11] to [13], the method further including removing a material inside the container without affecting the cleanliness level inside the container.

[15] The method according to any one of [11] to [14], in which the cell culture to be prepared is a cell culture for transplantation.

[16] The method according to [15], in which the cell culture for transplantation is a sheet-shaped cell culture.

[17] A cell culture for transplantation prepared by the method according to any one of [11] to [16].

In accordance with another aspect, a container for preparing a cell culture comprises: an interior surrounded by a wall and sealed from outside the container, with at least a part of the interior of the container having a cleanliness level within Class 1 to 9 specified in ISO 14644-1; an air filter positioned in the wall of the container, with the air filter being configured to permit air to enter and/or to exit the part of the interior of the container while maintaining the cleanliness level in the part of the interior of the container within the Class 1 to 9 specified in ISO 14644-1; at least one storage section positioned in the part of the interior of the container, with the at least one storage section storing material configured to be used for preparing the cell culture; and an elongated operating part that extends into the interior of the container and that is configured to be operated by a user positioned outside the container to manipulate the material stored in the at least one storage section during the preparing of the cell culture

Using a pouch-shaped container according to the disclosure here and, if necessary, a centrifuge, an incubator, or the like makes it possible to use a cell culture for transplantation without introducing a large facility. In other words, a cell culture for transplantation can be used not only in large hospitals which can make an investment in large facilities but also in hospitals provided with a space for deploying the container according to the present invention and a device such as a centrifuge and an incubator.

In addition, with the container disclosed here, a cell culture is kept inside the container with a clean space from the beginning of the preparation until being taken out when used. Accordingly, the cell culture has no chance to come into contact with the outside world, which reduces the risk of contamination.

### BRIEF DESCRIPTION OF THE DRAWINGS

FIG. 1 is a schematic view of a pouch-shaped container according to an embodiment disclosed by way of example.

FIG. 2A is a schematic view of the pouch-shaped container in a deflated state being stored in or positioned in a centrifuge bucket, and FIG. 2B is a side view of the pouch-shaped container in the deflated state before being positioned in the centrifuge.

### DETAILED DESCRIPTION

Set forth below with reference to the accompanying drawings is a detailed description of a sealed container for preparing a cell culture representing an example of the inventive sealed container disclosed here.

An aspect of the present disclosure relates to a container having a pouch shape used for preparing a cell culture.

In the present disclosure, a “cell culture” is prepared by cell culturing. The cell culturing herein may be adherent cell culturing or suspension cell culturing. Examples of the cell culture include, but are not limited to, a sheet-shaped cell culture, a cell culture for injection, and a cell culture in clumps. In an embodiment, the cell culture is used for cell transplantation. Preferable examples of the cell culture include a sheet-shaped cell culture for transplantation and a cultured cartilage (for example, JACC (registered trademark)).

The sheet-shaped cell culture herein has cells connected to each other to form a sheet. The cells may be directly connected to each other by, for example, a cell adhesion factor or may be indirectly connected via, for example, an extracellular matrix. The sheet-shaped cell culture may be a single layer or may have a multilayer structure with two layers, three layers, four layers or more. Alternatively, the sheet-shaped cell culture may have a three-dimensional structure with no layered structure.

In the present disclosure, the “container having a pouch shape” (pouch-shaped container) creates a sealed space that separates the inside of the container from the outside and has a size large enough to allow the preparation of a cell culture inside the container. In an embodiment, the container has an expandable/contractible (configured to be enlarged/reduced in size) structure. An example of the expandable/contractible structure herein includes, but is not limited to, an accordion container. In an embodiment, in order to maintain internal sealability, the container has a structure that hardly causes or is not likely to cause a hole or a rip when being used for preparing a cell culture. An example of the structure that hardly causes a hole or a rip includes, but is not limited to, a double structure. In an embodiment, the container includes or is fabricated from a freezable material. Examples of the freezable material include, but are not limited to, polyester, polyethylene, polypropylene, and polyurethane. Preferably, the freezable material is polyester. For example, a freezable container can be frozen together with a frozen cell (frozen cells) or the like stored inside the container.

Typically, an air filter is used to remove dust and dirt from air and to obtain clean air. Examples of the air filter include, but are not limited to, HEPA filters, ULPA filters, and gas removal filters which are used in air cleaners, clean benches, and the like.

In the present disclosure, the “air filter” is incorporated in the surface of the container and is the only part connecting the inside of the container and the outside world. When letting in air outside the container, the air filter removes fine particles and the like in the air and does not bring the particles into the container so that the particles are not introduced into the container. When letting out air inside the container, the air filter holds substances inside the container and does not release the substances to the outside world. With such a function, the air filter can keep the space inside the container clean. In addition, such a function enables the air filter not to contaminate the outside world with internal factors.

In an embodiment, the air filter enables the container to hold a high-cleanliness space inside. Preferably, the air filter keeps the space inside the container to have a cleanliness level ranging from Class 1 to 9 specified in ISO 14644-1 or JIS B 9920 or a cleanliness level ranging from Class 1 to 100,000 specified in US Federal Standard 209E. More preferably, the space inside the container has a cleanliness level ranging from Class 1 to 5 of ISO 14644-1. In an embodiment, the container may be manufactured in a high-cleanliness space, and the air filter may be configured to maintain the cleanliness level of the space inside the container as high as the level during manufacturing. In an embodiment, the air filter may be incorporated into an air purifier or the like to voluntarily enhance the cleanliness level of the space inside the container. In an embodiment, the air filter is a vent filter.

In an embodiment, air is drawn into the container via the air filter when the container is inflated. When air passes through the air filter, for example, dust and fine particles are removed, and the air is cleaned. In an embodiment, air inside the container is vented through the air filter when the container is deflated. When air passes through the air filter, the air filter removes factors inside the container so as not to take the factors outside.

In the present disclosure, an “operation section” is disposed on the surface of the container and enables operation inside the container from outside the container. In an embodiment, the operation section may have a glove-shaped structure protruding into the container and may allow a user to put his/her hands therein and to operate a part, an instrument, or the like inside the container. In an embodiment, the operation section may be deflated when the container is deflated and may become usable for the first time when the container is inflated and the space inside the container expands to such an extent as to expand the operation section.

In the present disclosure, a “storage section” is housed inside the container and configured to store a material to be used in the preparation of a cell culture or a waste liquid generated in the preparation. The storage section herein has an openable and closable structure to prevent the material stored inside from leaking into the container in the preparation of a cell culture. Examples of the material used in the preparation of a cell culture include, but are not limited to, a preparation medium, a cleaning liquid, a buffer solution, a cell suspension, a cell to be cultured, and an instrument used for preparing a cell culture. Known materials used for cell culturing can be employed as the preparation medium, cleaning liquid, and buffer solution. The “instrument used for preparing a cell culture” refers to any instrument used for preparing a cell culture except for the storage section itself. Examples of the instrument include, but are not limited to, pipettes, tips, droppers, tweezers, and spatulas. In an embodiment, the container may be provided with a plurality of storage sections.

The storage section may include one or more storage sections selected from the group consisting of a storage section functioning as “cell culture section”, a storage section functioning as “liquid storage section”, a storage section functioning as “centrifugation section”, a storage section functioning as “cell preservation section”, and a storage section functioning as “waste liquid section”. In an embodiment, the storage section may have a structure separable from the container without affecting the cleanliness level inside the container. The “structure separable from the container” refers to, for example, a structure in which the storage section can be separated without affecting the space inside the container when the space inside the container is separated from the storage section by heat sealing or the like from the outside of the container. In an embodiment, the storage section may store part of a cell suspension and may be separated from the container in order to measure a concentration of the cell suspension outside the container.

The “cell culture section” herein is a part for storing a seeded cell (seeded cells) and for preparing a cell culture. In an embodiment, the cell culture section may be a culture vessel. As the culture vessel, a known culture vessel used for cell culturing can be employed. Examples of the culture vessel include, but are not limited to, petri dishes, culture flasks, and microplates. As a preferred embodiment, the bottom surface of the cell culture section is coated with a culture substrate. As the culture substrate, a known substrate used for cell culturing can be employed. Examples of the culture substrate include, but are not limited to, collagen, fibronectin, and laminin. As a more preferred embodiment, the bottom surface of the cell culture section is coated with a temperature-responsive cell culture substrate. For example, UpCell (registered trademark) commercially available from CellSeed Inc. can be employed.

The “liquid storage section” herein is a storage section for storing various kinds of liquids other than a cell (cells) used for preparing a cell culture. In this section, for example, a medium, a cleaning liquid, and a buffer solution are stored. In an embodiment, the liquid storage section may have an openable and closable structure that can be sealed to prevent internal liquids from leaking out. An example of the structure includes a chuck.

The “centrifugation section” herein is a part having a structure suitable for storing a cell suspension and for centrifugation in the preparation of a cell culture. Herein, the “structure suitable for centrifugation” refers to, but is not limited to, a structure that is not damaged or deformed by a centrifugal force applied to the structure during centrifugation and has a pointed tip so as to easily separate a cell and supernatant after the centrifugation.

The “cell preservation section” herein is a part for storing a cell (cells) to be cultured before the preparation of a cell culture. In an embodiment, the cell preservation section may contain a lyophilized vial containing a cell inside the container.

The “waste liquid section” herein is a part for storing a waste liquid generated in the preparation of a cell culture. This section has a structure that does not allow the stored waste liquid to leak out of the waste liquid section and does not affect a liquid, a culture, or the like stored in other storage sections.

In an embodiment, the storage section may have a plurality of functions. For example, a certain storage section may double as a centrifugation section and a cell preservation section. That is, a storage section preserving a cell (cells) to be cultured may have a structure suitable for centrifugation. In this storage section, a cleaning liquid may be added to form a cell suspension, and the cell suspension may be subjected to centrifugation. For example, a storage section storing an instrument used for preparing a cell culture may be used as a waste liquid section after the instrument is taken out.

An aspect of the disclosure here relates to a kit for preparing a cell culture. The kit includes the aforementioned container having a pouch shape.

The kit may also include, for example, a cell or cells for preparing a cell culture, a preparation medium, a cleaning liquid, a buffer solution, a culture vessel, a centrifuge tube, a tube, an instrument used for cell culturing, a transport container, and instructions for use, but the contents of the kit are not limited thereto.

In another aspect, the disclosure here relates to a method for preparing a cell culture using a container having a pouch shape. The container is sealed and stretchable and has a cleanliness level that is maintained.

The term “stretchable” refers to ability of the container to inflate and/or deflate (expand/contract) without affecting internal conditions such as the cleanliness level.

In the present disclosure, the “preparing a cell culture” includes, but is not limited to: (i) warming and thawing a frozen cell (cells); (ii) transferring the cell (cells) into a centrifugation-resistant container; (iii) adding a cleaning liquid to the transferred cell (cells); (iv) performing centrifugation to separate supernatant from the cell (cells); (v) discarding the supernatant; (vi) adding a medium to the cell (cells), suspending the cell, and seeding the cell (cells) in a container suitable for culture; (vii) incubating the seeded cell (cells); (viii) using a buffer solution to wash the cell culture completed by the incubation in the previous step; and (ix) recovering the cell culture. Steps (iii) to (v) may be repeated multiple times after step (v) if necessary. Furthermore, if the cell (cells) is not frozen, steps (i) to (v) are unnecessary.

In an embodiment, the “preparing a cell culture” may be performed from step (vi) after step (i). In this case, components of a solution that preserves the frozen cell (cells) are removed by medium replacement.

In an embodiment, the “preparing a cell culture” may include a step of warming the container having a pouch shape, a step of placing the container in a bucket of a centrifuge, or a step of setting up the container in an incubator when the container is in a deflated state. In an embodiment, the “preparing a cell culture” may include a step of allowing an operation section and each storage section to be used when the container having a pouch shape is in an inflated state. For example, deflating the container reduces the occupied volume of the container, which makes it possible to house the container in an existing centrifuge, an incubator, or the like in the preparation of a cell culture. For example, inflating the container increases the volume of the container, which provides enough work space for preparing a cell culture.

In an embodiment, the “preparing a cell culture” may further include a step of removing a material inside the container without affecting the cleanliness level inside the container. For example, this step makes it possible to separate, from the container, the storage section storing a liquid used or the waste liquid section used. In addition, for example, a cell suspension obtained in steps (iii) to (v) can be taken out of the container by this step so as to measure a cell concentration.

Hereinafter, preferred embodiments of the disclosure here will be described in detail with reference to the drawings.

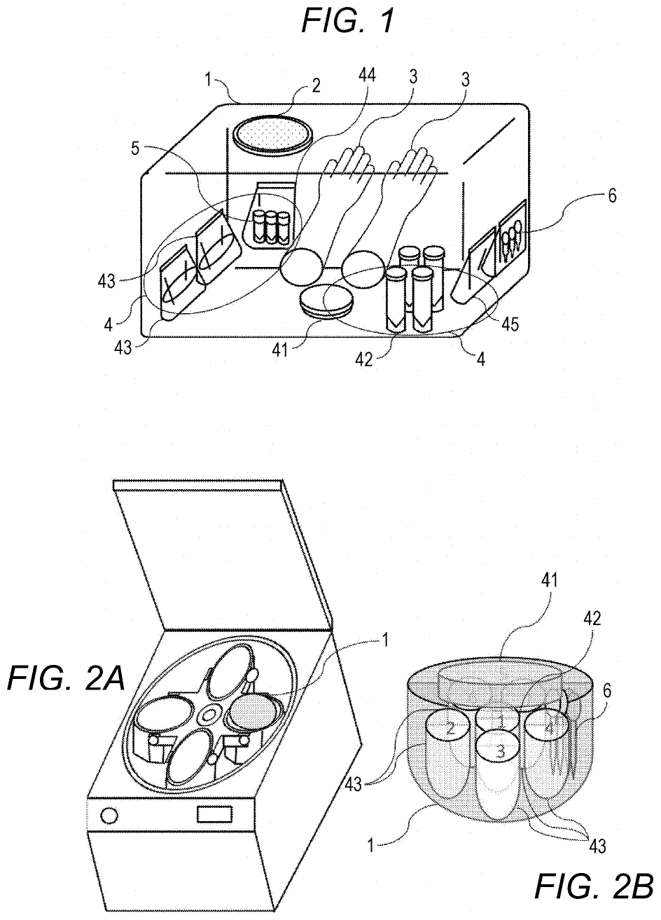

FIG. 1 is a conceptual diagram of a container according to one embodiment disclosed as an example of the inventive container. For the purpose of illustration, each drawing in this application shows the size and design of each part emphasized as appropriate. Accordingly, note that the size and design in each drawing are different from the actual ones.

As illustrated in FIG. 1, a container 1 according to one embodiment includes an air filter 2, an operation section 3, and storage sections 4. As shown in FIG. 1, the container includes a wall surrounding an interior in which is positioned the operation section 3 and the storage sections 4, and the air filter 2 is located in the wall of the container. The container (container interior) 1 may further include a cell (cells) 5 to be cultured and an instrument 6 used for preparing a cell culture. The operation section 3 allows a user to put his/her hands therein and to work with each storage section. That is, the user positioned outside the container 1 and using the operation section 3 is able to work with (manipulate) the storage sections positioned in the container interior. As shown in FIG. 1, the operation section 3 may be comprised of an elongated operating part(s) (glove-shaped) that extends into the interior of the container. The storage sections 4 optionally include a cell culture section 41, a centrifugation section 42, and liquid storage sections 43. In addition, the storage sections 4 optionally include a cell preservation section 44.

Next, an operation for preparing a cell culture with the container 1 will be described.

A user warms the container 1 which is shipped or deflated, thereby thawing a frozen cell (frozen cells) inside the cell preservation section 44. The container 1 is warmed, for example, by being immersed in a hot-water bath at constant temperature of 37° C. Deflating, and then, warming the container make it possible to downsize a warming device and to easily transfer heat to the frozen cell. Next, the container 1 is inflated to expand a space inside the container. Accordingly, the operation section 3 and each storage section 4 can be used.

The user puts his/her hands in the operation section 3 and moves the thawed, previously frozen, cell(s) to the centrifugation section 42. Furthermore, a cleaning liquid stored in any one of the liquid storage sections 43 (hereinafter referred to as “cleaning liquid”) is added to the cell(s). If necessary, the cleaning liquid is added in stages.

The user deflates the container 1 so that the container exhibits a deflated configuration such as shown in FIG. 2B, places the deflated container in a bucket of a centrifuge, and performs centrifugation. FIG. 2A is a schematic view when the deflated container 1 is stored in or positioned in a centrifuge. Deflating the container reduces the size of the container to such an extent as to be placed in an existing centrifuge bucket. Accordingly, the existing centrifuge can be used.

After the centrifugation, the container 1 is inflated so that the operation section 3 and each storage section 4 can be used, and supernatant in the centrifugation section 42 is discarded in a waste liquid section 45 through use of the operation section 3. Furthermore, the cleaning liquid is added, and the centrifugation is repeated.

After the last centrifugation, the container 1 is inflated again, supernatant is discarded, and then, a preparation medium stored in any one of the liquid storage sections 43 is added to the cell (cells) and suspended.

Before the last centrifugation, a cell suspension is included in one of the storage sections 4 that is separable from the container 1 and then separated not to affect the cleanliness inside the container. Accordingly, the cell suspension is taken out of the container, and a cell concentration of the cell suspension is measured, thereby calculating an amount of the cell suspension added to the preparation medium. For example, the cell concentration may be measured outside the container using a hemocytometer.

In the cell culture section 41, the user seeds the cell(s) suspended in the preparation medium. After that, the container 1 is deflated, and the deflated container 1 is set up in an incubator for culturing. For example, the cell is cultured in a CO₂ incubator for 2 to 26 hours. At this time, deflating the container reduces the occupied volume of the container inside the incubator, which makes it possible to set up a plurality of containers in one incubator.

On completion of the culturing, culture supernatant is discarded in the waste liquid section 45, and the cell (cells) is washed with a buffer solution stored in any one of the liquid storage sections 43. The washing is performed several times as necessary.

A cell culture is allowed to stand as necessary, and then, the container 1 is opened in a high-cleanliness room such as an operating room, thereby collecting the cell culture.

Using the container disclosed here makes it possible to prepare a cell culture without introducing a large-scale facility for preparing a high-cleanliness space. Accordingly, it is not necessary to make a heavy investment for introducing a large-scale facility, and it is possible even for a medium-scale hospital to use a cell culture for transplantation having a short use-by date, thereby widely disseminating regenerative medicine using a cell culture for transplantation.

The detailed description above describes an embodiment of a sealed pouch-shaped container and operational method representing an example of the inventive sealed pouch-shaped container and operational method disclosed here. The invention is not limited, however, to the precise embodiment and variations described. Various changes, modifications and equivalents can be effected by one skilled in the art without departing from the spirit and scope of the invention as defined in the accompanying claims. All such changes, modifications and equivalents which fall within the scope of the claims are expressly intended to be embraced by the claims.

What is claimed is:

1. A container for preparing a cell culture, the container comprising:
an interior surrounded by a wall and sealed from outside the container, at least a part of the interior of the container having a cleanliness level within Class 1 to 9 specified in ISO 14644-1;
an air filter positioned in the wall of the container, the air filter being configured to permit air to enter and/or to exit the part of the interior of the container while maintaining the cleanliness level in the part of the interior of the container within the Class 1 to 9 specified in ISO 14644-1;
at least one storage section positioned in the part of the interior of the container, the at least one storage section storing material configured to be used for preparing the cell culture; and
an elongated operating part that extends into the interior of the container and that is configured to be operated by a user positioned outside the container to manipulate the material stored in the at least one storage section during the preparing of the cell culture.

2. The container according to claim 1, wherein the elongated operating part is an elongated glove-shaped operating part.

3. The container according to claim 1, wherein the container is collapsible.

4. A pouch-shaped container, the pouch-shaped being sealed and comprising:
an air filter configured to maintain a space inside the container to have a cleanliness level within a predetermined range of Class 1 to 9 specified in ISO 14644-1;
at least one storage section housed inside the container, the at least one storage section being configured to store material to be used for preparing a cell culture; and
an operation section configured to allow manipulation of the material to be used for preparing a cell culture from outside the container.

5. The container according to claim 4, wherein the container is a container that is configured to be expanded and contracted.

6. The container according to claim 4, wherein the container is fabricated from a freezable material.

7. The container according to claim 4, wherein the operation section comprises a glove-shaped structure protruding into the container.

8. The container according to claim 4,
wherein the storage section includes at least one selected from the group consisting of:
a cell culture section configured to store a seeded cell and to culture the cell,
a liquid storage section configured to store a plurality of liquids,
a centrifugation section configured to store a cell suspension to be centrifuged,
a cell preservation section configured to store a cell to be cultured, and
a waste liquid section configured to store a waste liquid.

9. The container according to claim 4, wherein the storage section has a structure separable from the container.

10. The container according to claim 8, wherein the storage section includes the cell culture section, and the cell culture section includes a temperature-responsive cell culture substrate.

11. A kit for preparing a cell culture, the kit comprising the container according to claim 4.

12. The kit for preparing a cell culture according to claim 11, the kit further comprising a culture vessel having a temperature-responsive cell culture substrate.

13. The kit for preparing a cell culture according to claim 11, the kit further comprising an instrument used for preparing a cell culture.

14. A method for preparing a cell culture inside a container from outside the container, the method comprising:
seeding a cell to be cultured in a space inside the container while the container is sealed, the container being a pouch-shaped container and the space inside the container having a cleanliness level kept within a predetermined range specified in ISO 14644-1 during the seeding of the cell; and
culturing the cell in the space in the container.

15. The method for preparing a cell culture according to claim 14, further comprising a) warming the pouch-shaped container h, b) placing the container in a bucket of a centrifuge, or c) setting up the container in an incubator when the container is in a deflated state.

16. The method for preparing a cell culture according to claim 14, the method comprising allowing an operation section and each storage section to be used when the container having a pouched shape is in an inflated state.

17. The method according to claim 14, the method further comprising removing a material inside the container without affecting the cleanliness level inside the container.

18. The method according to claim 14, wherein the cell culture is a cell culture for transplantation.

19. The method according to claim 18, wherein the cell culture for transplantation is a sheet-shaped cell culture.

20. A cell culture for transplantation prepared by the method according to claim 14.
